(19) United States
(12) Patent Application Publication
(10) Pub. No.: US 2005/0228403 A1
(43) Pub. Date: Oct. 13, 2005

(54) Tissue cutting devices and methods

(75) Inventors: Huddee Jacob Ho, San Jose, CA (US); Roberta Lee, Redwood City, CA (US); Samuel E. Zuckswert, San Jose, CA (US)

Correspondence Address: Jung-hua Kuo;Attorney At Law, PO Box 3275, Los Altos, CA, 94024, US

(73) Assignee: MANOA MEDICAL, INC., A DELAWARE CORPORATION, Redwood City, CA (US)

(21) Appl. No.: 10/815,912

(22) Filed: Mar. 31, 2004

(51) Int. Cl. A61B 17/32

(52) U.S. Cl. 606/113

(57) ABSTRACT

Minimally invasive devices and methods for cutting a volume of soft tissue such as a biopsy or a therapeutic excision of cancer are disclosed. The device generally includes a probe, a cutting loop with sufficient elasticity, shape memory or superelastic property such that the loop returns to a cutting configuration when released from a storage configuration, and a loop holder to hold and rotate the cutting loop about a loop holder axis when the cutting loop is in the cutting configuration so as to adjust a loop angle between the probe axis and the cutting loop. The method generally includes positioning the tissue cutting device adjacent the volume of tissue, releasing the cutting loop from the storage configuration to the cutting configuration, rotating the cutting loop to adjust the loop angle, and moving the tissue cutting device to cut the volume of tissue.

### BACKGROUND OF THE INVENTION

1. Field of the Invention

The present invention relates generally to devices and methods for cutting a volume of soft tissue. More specifically, minimally invasive devices and methods for cutting a volume of soft tissue such as a biopsy or a therapeutic excision of cancer are disclosed.

2. Description of Related Art

Minimally invasive procedures have instigated a need for refinement in surgical devices that can function within confined spaces, particularly in soft tissue, such as breast tissue. Devices that are typically used during open surgical procedures (i.e. scalpel, scissors, electrosurgical “pencil” electrodes) are often not adaptable for use in a minimally invasive procedure. Furthermore, minimally invasive procedures cannot be directly visualized as the skin incision is typically just large enough to insert the surgical device and are therefore often guided by medical imaging or by video camera as during laparoscopy. In the breast, mammography, ultrasound and magnetic resonance imaging (MRI) are used to guide minimally invasive procedures. Current surgical devices that use an oscillating sharp edge or radio frequency energy to cut the tissue retrieve a specimen of generally fixed volume and are not adaptable to excise lesions of different or asymmetric volumes. Breast cancer grows within the milk duct(s), or towards the skin in Cooper's ligament in addition to growing outward in a radial direction as a mass. Current minimally invasive devices are designed to excise the mass and are not adaptable for excision of an associated diseased duct(s) or Cooper's ligament. Leaving cancer behind in the duct(s) and/or in Cooper's ligament increases the risk of local recurrence despite the administration of post operative radiation or other adjuvant therapy.

Open surgical biopsy removes lesions of variable or irregular volume but an excessive amount of normal breast tissue is often also removed leading to a poor cosmetic result. In addition, open surgical biopsy typically requires a significant skin incision resulting in a longer, permanent scar. More importantly, a diseased duct(s) containing cancerous cells is not detectable by direct vision or by palpation during an open surgical procedure. Although the main cancerous mass may be excised, a diseased duct(s) is not identifiable during the procedure and may unintentionally not be fully included in the specimen.

Axial ductal ultrasound is a method of ultrasound scanning of the breast that demonstrates the internal anatomy of the breast. In particular, the milk ducts and lobes of the breast are identified resulting in visualization of not only a lesion but also a diseased duct(s) and extension of the cancer into Cooper's ligament. Multifocal cancers or additional cancers associated with the diseased duct may also be visualized. Therefore, the entire disease process (i.e. the lesion and extensions of the lesion within the breast) is visualized and can be removed under direct, real-time ultrasound guidance.

Devices to excise a volume of soft tissue in the breast typically are designed to remove a fixed volume of tissue and are not designed to remove a long segment of tissue such as a diseased milk duct. Repetitive insertions and removals of the device would be required to fully excise the entire disease process.

U.S. Pat. No. 6,575,970 to Quick describes a shaft rotatably mounted to a probe at an angle and an arcuate cutting surface secured to the shaft. The length of the shaft is longer in dimension than a probe width and defines the diameter of the arcuate cutting surface. The shaft is rotatable causing the arcuate cutting surface to rotate. This device requires a skin incision that is at least as long as the length of the shaft to enter the tissue and is not amenable for use through a small skin incision.

What is needed is a device and method for a minimally invasive procedure that is capable of excising a lesion of variable dimensions within a single volume of tissue from a breast or other soft tissue. More specifically, there is a need for a device and method to excise or biopsy a disease process within a breast that includes not only the main focus of the disease (i.e. a lesion or a mass) but also the milk duct or ducts that are also affected and any other growth of the disease (e.g. growth into Cooper's ligament). Preferably the procedure can be guided using medical imaging.

### SUMMARY OF THE INVENTION

Minimally invasive devices and methods for cutting a volume of soft tissue such as a biopsy or a therapeutic excision of cancer are disclosed. It should be appreciated that the present invention can be implemented in numerous ways, including as a process, an apparatus, a system, a device, or a method. Several inventive embodiments of the present invention are described below.

The tissue cutting device for excising a volume of soft tissue comprises a handle, a probe, a loop holder and a cutting loop. The loop holder is housed within the probe and is extendable and retractable with respect to the probe. The cutting loop is attached to the loop holder and has a loop shape that defines a loop shape width and a loop shape height. The cutting loop is flexible such that the loop shape is variable depending on the presence of one or more external stresses placed on the cutting loop. The loop holder has a length that is generally less than a width of the loop shape width.

The cutting loop is preferably made from a metal or metal alloy having sufficiently high elasticity, superelastic properties and/or shape memory capability to facilitate insertion of the probe and cutting loop into the tissue through a small incision. The cutting loop preferably comprises a single loop. In an alternative, the cutting loop is comprised of more than one loop which for simplification purposes is described herein as a cutting loop. The more than one loop is configured from the same or different materials.

The probe has a length defining a probe axis and a distal end. The loop shape height defines a loop axis. The angle between the loop axis relative to the probe axis is variable. When the probe is penetrating into soft tissue during positioning, the cutting loop is in a penetrating configuration where the loop axis is configured to align at an angle that is generally 0° relative to the probe axis to facilitate ease of penetration. During insertion the cutting loop is preferably housed within the confines of the probe. After the probe is positioned in the tissue in the desired location, the cutting loop is advanced out of the distal end such that the cutting loop returns to a preformed, generally circular primary loop shape configuration due to the high elasticity, or superelastic property of the material used to configure the cutting loop. Furthermore, the high elasticity or superelastic property of the material prevents permanent deformation of the cutting loop when at least partially housed within the probe. The cutting loop is rotatable relative to the probe axis to vary the angle between the loop axis and the probe axis from generally 0° to 180°. To facilitate cutting of soft tissue, the cutting loop may have one or more sharpened edges. Furthermore, the cutting loop may be energized such as with radio frequency energy and/or the loop may be configured to oscillate along a predetermined or variable distance, direction and/or frequency. The loop shape may be fixed or variable by adjusting the width and/or height of the loop.

A method for cutting a volume of soft tissue generally includes identifying a lesion in the tissue with an targeting device and determining an estimated volume of tissue to be excised that includes at least a part of the lesion for diagnostic sampling. For a therapeutic excision, the estimated volume of tissue to be excised preferably includes the entire lesion and a surrounding margin of normal tissue. More specifically in the breast, the volume of soft tissue contains at least one of a lesion, a duct or ducts, a Cooper's ligament and a lobe or part of a lobe. Preferably, the probe is positioned in the tissue adjacent to the targeted volume of tissue with the cutting loop in the penetrating configuration. Energy such as radio frequency energy and/or oscillation may be used to facilitate tissue penetration. Once the probe is positioned in the desired location the cutting loop is advanced through a distal end of the probe. The cutting loop is energized and rotated from the penetrating configuration to a cutting configuration. After the cutting loop is in the cutting configuration, the probe is advanced or retracted moving the cutting loop along a length of the cut to create or complete a circumferential cut around the volume of tissue. In one embodiment the primary loop shape of the cutting loop determines the loop shape width and loop shape height. The width of the volume of tissue being cut is predetermined but the height of the volume of tissue is varied by varying the amount of rotation of the cutting loop in the cutting configuration. In an alternative, the cutting loop is expandable and/or retractable in loop shape width and/or loop shape height to accommodate variations in the desired volume of tissue being excised. During the positioning of the probe and/or the cut, the cutting loop may be energized from an external energy source (e.g. radio frequency energy) and/or may oscillate. Oscillation of the cutting loop is preferably independent of the probe advancement or retraction and may be in one of several directions. Once on the opposite side of the volume of tissue from where the cut was initiated, the cutting loop is rotated to the 0° or 180° position relative to the probe axis to complete the cut. In a further embodiment, after the cutting loop has rotated to the 180° position, the cutting loop is released from a rotating control mechanism but not detached from the tissue cutting device and passively moves to a position(s) of least resistance as the probe is removed from the tissue.

The procedure is preferably guided using a targeting device. Preferably the targeting device is an imaging device. The imaging device is one of external to the patient and within the patient. When inserted into the tissue the imaging device is one of incorporated or attached to the probe and separate from the probe. In one embodiment, the probe contains one or more locators that provide additional means of identifying preferably the distal end of the probe within the tissue.

These and other features and advantages of the present invention will be presented in more detail in the following detailed description and the accompanying figures which illustrate by way of example principles of the invention.

### BRIEF DESCRIPTION OF THE DRAWINGS

The present invention will be readily understood by the following detailed description in conjunction with the accompanying drawings, wherein like reference numerals designate like structural elements.

FIGS. 1A and 1B are perspective views and FIGS. 1C-1F are top views of exemplary embodiments of a tissue cutting device with a cutting loop in the penetrating and advanced configurations.

FIGS. 2A-2C are perspective views illustrating the cutting loop in the cutting configuration.

FIG. 2D is a top view of a handle.

FIGS. 2E and 2F are a top view and a cross-sectional side view, respectively, of an exemplary embodiment of the tissue cutting device.

FIG. 3A is a perspective view illustrating a part of the cutting loop in the cutting configuration.

FIGS. 3B-3F are partial side views of additional embodiments of the cutting loop in the cutting configuration.

FIG. 4A and FIG. 4B are cross-sectional side and front views, respectively, of an embodiment of the tissue cutting device illustrating a mechanism of oscillation of the cutting loop.

FIGS. 5A-5C are top views of embodiments of the cutting loop.

FIGS. 6A and 6B are top views of further embodiments of the cutting loop.

FIG. 7 is a perspective view of an exemplary specimen of tissue.

FIGS. 8A-8D are perspective views illustrating a method of excising a volume of tissue using the tissue cutting device.

FIG. 9 is a flowchart illustrating a method of excising a volume of tissue.

### DESCRIPTION OF SPECIFIC EMBODIMENTS

Minimally invasive devices and methods for cutting a volume of soft tissue such as a biopsy or a therapeutic excision of cancer are disclosed. The following description is presented to enable any person skilled in the art to make and use the invention. Descriptions of specific embodiments and applications are provided only as examples and various modifications will be readily apparent to those skilled in the art. The general principles defined herein may be applied to other embodiments and applications without departing from the spirit and scope of the invention. Thus, the present invention is to be accorded the widest scope encompassing numerous alternatives, modifications and equivalents consistent with the principles and features disclosed herein. For purpose of clarity, details relating to technical material that is known in the technical fields related to the invention have not been described in detail so as not to unnecessarily obscure the present invention.

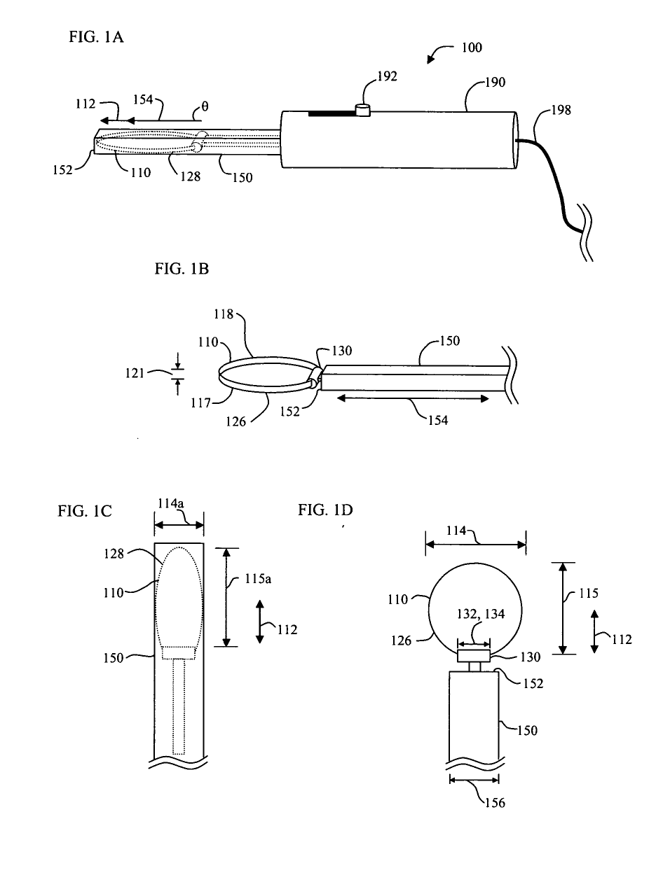

FIGS. 1A-1D illustrate an embodiment of a tissue cutting device 100 generally including a probe 150 extending from a handle 190 and a cutting loop 110 affixed to a loop holder 130. The probe 150 has a distal end 152, a probe width 156 and a length that defines a probe axis 154. The loop holder 130 has a loop holder length 132 that defines a loop holder axis 134 generally orthogonal to the probe axis 154. The loop holder length 132 is preferably of smaller dimension than the probe width 156 to permit the loop holder 130 to advance and retract within the probe 150 along the probe axis 154. Although not shown, the probe 150 may optionally contain one or more accessory channels or lumens that communicate with one or more ports located on the handle 190 or a proximal region of the probe 150. The channels may enable passage of fluid such as an anesthetic or an irrigation fluid to the tissue near the cutting loop 110 and/or provide a vacuum created by an external vacuum source to evacuate fluids from the tissue near the cutting loop 110.

The cutting loop 110 may be formed of a metal, a metal alloy, ceramic, glass, plastic and/or a polymer, for example. Preferably, the cutting loop 110 is made of a material that has shape memory properties and/or superelastic properties such as a nickel titanium alloy (i.e., NiTi or nitinol), and/or a material with a sufficiently high elasticity. In one embodiment, the cutting loop 110 may be formed of an electrically conductive material such as a metal, metal alloy, metal laminate, and/or metal composite. For example, the metallic material may be titanium, titanium alloy, nickel-titanium alloy, nickel-chromium alloy, chromium-nickel alloy, cobalt chromium-nickel alloy and/or iron-chromium alloy. Preferably the cutting loop 110 is preformed to a primary loop shape (i.e., a cutting configuration) 126 as shown in FIGS. 1B and 1D, the method of which is well known to those skilled in the art. The primary loop shape 126 defines a primary loop shape width 114 and a primary loop shape height 115 and defines at least part of a circle, an oval, a triangle, a square, a rectangle, a polygon or any other suitable shape that optimizes the cutting of soft tissue in general or for a specific procedure depending on the application of the tissue cutting device 100.

Upon application of one or more external stresses, the high elasticity or superelastic property of the cutting loop 110 allow the cutting loop 110 to reconfigure to a secondary loop shape (i.e., a non-cutting or storage configuration) 128 without the development of a permanent deformity as long as the resulting strains do not exceed the recoverable strain limits of the material of the cutting loop 110. When the external stress(es) is removed, the cutting loop 110 preferably generally returns to the primary loop shape 126.

As shown in FIG. 1A and in a top view in FIG. 1C, the cutting loop 110 can be housed within the probe 150. The internal walls of the probe 150 apply sufficient external stress to cause the cutting loop 110 to reconfigure to the secondary loop shape 128 defining a secondary loop shape width 114a and a secondary loop shape height 1115a. The secondary loop shape width 114a is generally smaller in dimension than the primary loop shape width 114 and the secondary loop shape height 115a is generally longer in dimension than the primary loop shape height 115. When the tissue cutting device 100 is passed through a skin incision into the tissue, the size of the skin incision needed is smaller when the cutting loop 110 is in the secondary loop shape 128 than if the cutting loop 110 were in the primary loop shape 126. The cutting loop 110 in the secondary loop shape 128 providing a smaller profile for the probe 150 and cutting loop 110 combination also facilitates positioning of the probe 150 within the tissue.

When the cutting loop 110 and the loop holder 130 are advanced through the distal end 152 of the probe 150 as shown in FIG. 1B and in a top view in FIG. 1D, the cutting loop 110 returns to the primary loop shape 126. Movement of the loop holder 130 along the probe axis 154 is controlled by a loop controller 192 located in the handle 190. In an alternative embodiment, as illustrated in the top views in FIGS. 1E and 1F, a probe cover 158 encompasses at least part of the probe 150 and is slidable along at least a portion of the length of the probe 150. Preferably there is a catch mechanism (not shown) to prevent the probe cover 158 from being completely detached from the probe 150. When the probe cover 158 is at or near at least part of the distal end 152 of the probe 150, the probe cover 158 houses at least part of the loop holder 130 and the cutting loop 110 reconfiguring at least part of the cutting loop 110 into the secondary loop shape 128 as shown in FIG. 1E. As shown in FIG. 1F, when the probe cover 158 is retracted at least partially towards the handle 190, the loop holder 130 and cutting loop 110 are exposed and the cutting loop 110 returns to the primary loop shape 126. Although not shown, a sheath may be placed in the tissue such that the probe cover 158 housing the cutting loop 110 in the secondary loop shape 128 catches or affixes to an external proximal end of the sheath. As the tissue cutting device 100 is advanced along the probe axis 154, the probe cover 158 is pushed against and remains generally stationary relative to the sheath while the cutting loop 110, the loop holder 130 and a distal portion of the probe 150 are advanced through the probe cover 158 and the sheath. Once the cutting loop 110 and the loop holder 130 have been advanced past a distal end of the sheath, the cutting loop 110 returns to its primary loop shape 126. The loop holder length 132 is preferably less than a width of the probe cover 158 and the sheath.

The cross-sectional area of the cutting loop 110 may define at least part of a circle, oval, diamond, triangle, rectangle, square, any other polygon and/or any combination of various shapes. Referring again to FIG. 1B, the cutting loop 110 has a leading edge 118 and a trailing edge 117. The leading edge 118 and/or the trailing edge 117 may be pointed, flat, rounded, dull, sharpened and/or serrated. The serrations may be continuous, intermittent, regular and/or irregular. The leading edge 118 and the trailing edge 117 may be configured using various methods such as chemical etching, machining and/or lasering. The leading edge 118 and/or the trailing edge 117 facilitates in separating and/or cutting the tissue. The distance between the leading edge 118 and the trailing edge 117 defines a loop width 121 which may be constant or variable along a length of the cutting loop 110.

The cutting loop 110 may be energized using radio frequency, laser, ultrasound, heat, cold, oscillation, vibration, rotation, and/or liquid and/or gas pressure. The cutting loop 110 may be operatively coupled to an external energy source (not shown) using a connector 198. In an alternative, the energy source (not shown) may be housed within the handle 190. When the cutting loop 110 is energized by radio frequency energy, the cutting loop 110 is configured as a monopolar or a bipolar electrode.

The cutting loop 110 may be at least partially include one or more additional materials. The additional materials may be configured as one or more layers, portions or segments that are continuous or discontinuous, symmetric or asymmetric, on the surface or within the cutting loop 110. The additional materials may provide properties such as electrical and/or heat insulation, increased electrical and/or heat conductivity, strength, lubricity, and sensors. The additional material(s) may include ceramics, polymers, plastics, metals, metal alloys, glass, diamonds, diamond-like carbon, diamond noncomposite coating (metal-doped or nonmetal-doped) and/or various other substances. Preferably when radio frequency energy is used as the external energy source, the cutting loop 110 is at least partially covered with an insulating material to concentrate the cutting current on the leading edge 118 and/or the trailing edge 117. The insulating material is preferably of sufficient dielectric strength to prevent dissipation of the cutting current into the tissue and to concentrate the cutting current at the leading edge 118 and/or the trailing edge 117.

The cutting loop 110 may include one or multiple loops. The multiple loops of the cutting loop 110 may have similar or dissimilar properties, configurations and/or functions. In one embodiment (not shown), the cutting loop 110 is comprised of an outer and an inner loop. The inner loop is nested within the outer loop. Preferably the leading edges 118 and/or the trailing edges 117 of the inner and outer loops are serrated. The inner loop oscillates and/or rotates to cut tissue. The outer loop oscillates and/or rotates in an opposing direction to the inner loop which facilitates cutting by preventing the tissue from moving with the oscillation or rotation of the inner loop. In an alternative, one or the outer loop and the inner loop does not oscillate or rotate and facilitates stabilization of the tissue.

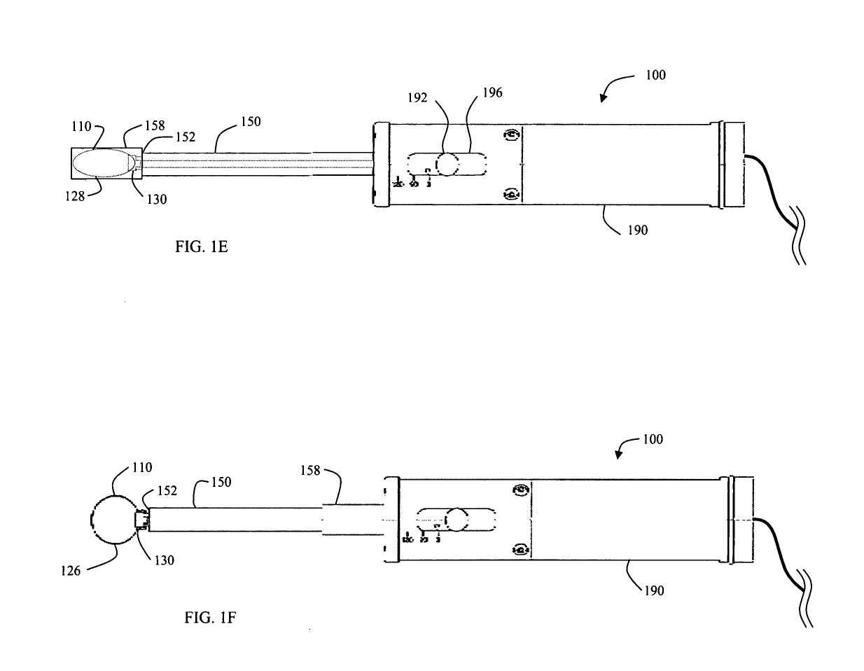

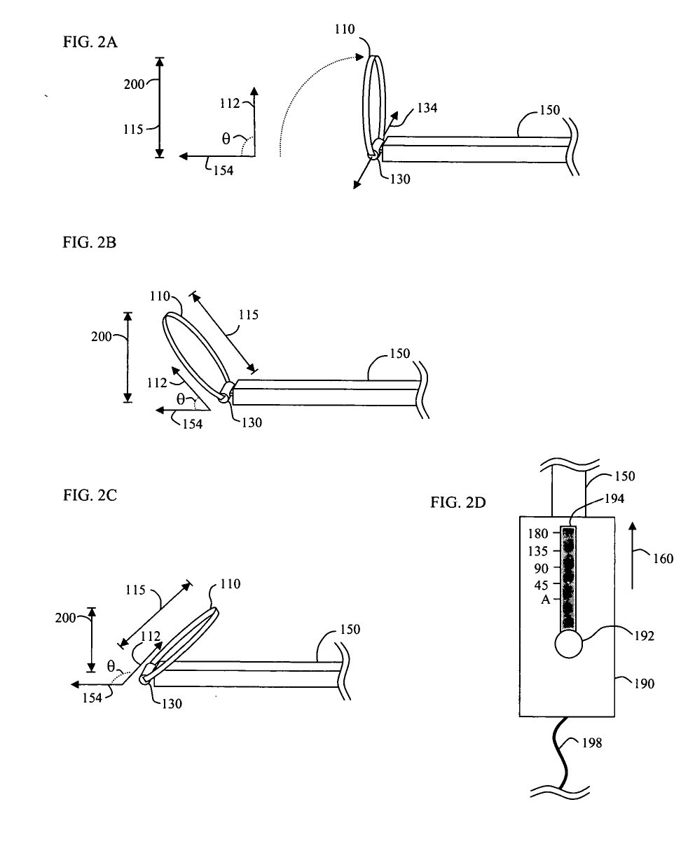

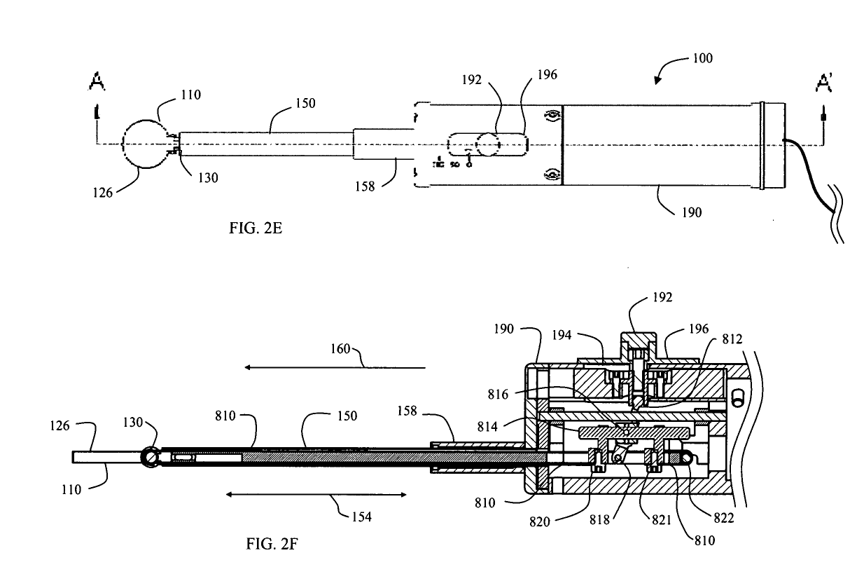

As shown in FIGS. 1A-1D, the primary loop shape height 115 and the secondary loop shape height 115a define a loop axis 112. The relation between the loop axis 112 and the probe axis 154 defines a loop angle θ. When the cutting loop 110 is in the secondary loop shape 128 and the loop angle θ is approximately 0° as shown in FIG. 1A, the cutting loop 110 is in a penetrating configuration. When the cutting loop 110 and the loop holder 130 are not housed within the probe 150 or the probe cover 158, the loop holder 130 may be rotatable about the loop holder axis 134 as shown in various views of the embodiment illustrated in FIGS. 2A-2C. Rotation of the loop holder 130 controls rotation of the cutting loop 110. When the cutting loop 110 has rotated such that the loop angle θ is greater than 0° and less than 180°, the cutting loop 110 is in a cutting configuration. In FIG. 2A, the cutting loop 110 has rotated to the loop angle θ of approximately 90°. When the loop angle θ is approximately 90°, a cut height 200 defined as the vertical dimension of a tissue specimen 620 that is cut by the cutting loop 110 as illustrated in FIG. 7, is generally the same as the loop shape height 115. In FIGS. 2B and 2C, the cutting loop 110 is rotated such that the loop angle θ is between 0° and 90° and between 90° and 180°, respectively, such that the cut height 200 is less than the loop height 115 and the cut height 200 is determined by the loop angle θ and the loop height 115, e.g., loop height 115×sin θ.

FIG. 2D is a top view of the handle 190 illustrating an exemplary embodiment of the loop controller 192 when the cutting loop 110 and the loop holder 130 (not shown) are initially housed within the probe 150. The loop controller 192 is slidable within a slot 194. When the loop controller 192 is manually moved to a position A located along the slot 194, the loop holder 130 and cutting loop 110 advance out of the distal end 152 of the probe 150 (not shown) and the loop angle θ stays at generally 0°. When the loop controller 192 is moved further to a position 45, the loop holder 130 and cutting loop 110 rotate such that the loop angle θ is generally 45°. When the loop controller 192 is moved to a position 90, the loop angle θ is generally 90°. The loop controller 192 at a position 135 corresponds to the loop angle θ of generally 135° and the loop controller 192 at a position 180 corresponds to the loop angle θ of generally 180°. Preferably, the loop holder 130 and cutting loop 110 are rotated such that the loop angle θ is greater than 0° and less than 180° as the probe 150 is advanced or retracted to cut along a specimen length 630 as shown in FIG. 7. The mechanism of rotating the loop holder 130 may employ the use of cables, rods, cams, pistons, rollers and/or gears.

An alternative embodiment illustrating a mechanism for rotation of the loop holder 130 when a probe cover 158 initially houses the loop holder 130 and the cutting loop 110 is shown in a top view in FIG. 2E and in a cross-sectional side view in FIG. 2F taken along line A-A′ in FIG. 2E. The loop holder 130 and the cutting loop 110 are rotatable only after the probe cover 158 is sufficiently retracted towards the handle 190 such that the cutting loop 110 returns to the primary loop shape 126 and the loop holder 130 is sufficiently exposed to permit rotation. The loop controller 192 is manually slidable within the slot 194. Affixed to and slidable with the loop controller 192 is a slot cover 196 that covers the slot 194 and prevents foreign substances (e.g. liquid) from entering the slot 194. The loop controller 192 controls a lever arm 812 such that movement of the loop controller 192 causes the lever arm 812 to rotate around a hinge 818. A driving point 816 mechanically affixes the lever arm 812 to a cable driver 814. Movement of the lever arm 812 around the hinge 818 causes the cable driver 814 to move along the probe axis 154 in a direction similar to the direction of movement of the loop controller 192. A cable 810 at least partially encircles the loop holder 130 and extends within the probe 150 to at least partially encircle a cable wheel 822 located in the handle 190. The ends of the cable 810 are affixed to cable fasteners 820 and 821 located on the cable driver 814. Movement of the cable driver 814 in the direction 160 pulls the segment of cable 810 attached to the cable fastener 821 in the direction 160 causing the entire cable 810 to move in a clockwise direction in the orientation shown in FIG. 2F which rotates the loop holder 130 and cutting loop 110 to a loop angle θ greater than 0° and less than or equal to 180° depending on the amount of rotation. Similarly, movement of the cable driver 814 in a direction opposite to direction 160 causes the cable 810 to move in a counterclockwise direction in the orientation shown in FIG. 2F which decreases the loop angle θ. The components described herein (e.g. cable driver 814) are described as a single unit but may be multiple units. Although one mechanism is described, various other suitable mechanisms that can implement rotation of the cutting loop 110 may be employed. In a further embodiment (not shown), the cutting loop 110 may be operatively uncoupled from the loop controller 192 and not disconnected from the tissue cutting device 100 preferably after completion of cutting of a specimen. Uncoupling of the cutting loop 110 from the loop controller 192 allows the cutting loop 110 to move to one or more positions of least resistance to facilitate removal of the probe 150 and the cutting loop 110 from the tissue.

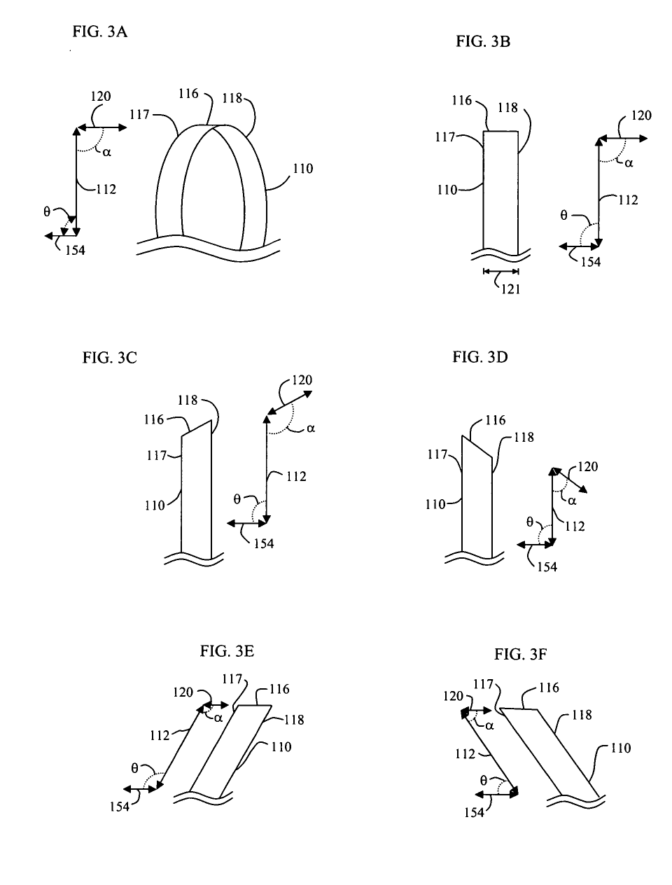

FIGS. 3A and side views in FIGS. 3B-3F illustrate various embodiments of the cutting loop 110. The cutting loop 110 has a loop peak 116. The relation of the leading edge 118 to the trailing edge 117 at the loop peak 116 defines a peak axis 120. The peak axis 120 and the loop axis 112 define an edge angle α. As shown in FIGS. 3A and 3B, when the cutting loop 110 is configured such that a length of the leading edge 118 is generally equal to a length of the trailing edge 117, the edge angle α is generally 90°. When the length of the leading edge 118 is greater than the length of the trailing edge 117, the edge angle α is greater than 90° as shown in FIG. 3C and when the length of the leading edge 118 is less than the length of the trailing edge 117, the edge angle α is less than 90° as shown in FIG. 3D.

Preferably the cutting loop 110 is rotated to a position during cutting along the specimen length 630 (shown in FIG. 7) such that the loop angle θ is generally equal to the edge angle α. When the loop angle θ and the edge angle α are generally equal, the peak axis 120 is generally parallel to the probe axis 154 such that the leading edge 118 at the loop peak 116 cuts tissue in a direction that is generally parallel to the probe axis 154. In FIG. 3E, the cutting loop 110 is configured such that the length of the leading edge 118 is greater than the length of the trailing edge 117 corresponding to the embodiment of the cutting loop 110 illustrated in FIG. 3C. In FIG. 3F, the cutting loop 110 is configured such that the length of the leading edge 118 is less than the length of the trailing edge 117 corresponding to the embodiment of the cutting loop 110 illustrated in FIG. 3D. In the embodiments illustrated in FIGS. 3E and 3F, the cutting loop 110 is rotated such that the loop angle θ is generally equal to the edge angle α which causes the leading edge 118 at the loop peak 116 to cut tissue generally parallel to the probe axis 154.

In a further embodiment, the cutting loop 110 oscillates and/or rotates in a direction preferably orthogonal to the direction of the cut during the cutting of tissue. The frequency of oscillation and/or rotation can be slow, e.g. approximately 1 Hz to 25 Hz, medium, e.g. between approximately 25 Hz to 50 Hz, and fast, e.g. greater than approximately 50 Hz. The peak-to-peak distance of oscillation may be predetermined or variable. Preferably, the peak-to-peak distance is approximately 1 to 10 mm although the peak-to-peak distance may be less than 1 mm or greater than 10 mm. Oscillation and/or rotation facilitates cutting of soft tissue, for example, by preventing eschar build-up on the cutting loop 110 when radio frequency energy is used and by improving the cutting mechanism if the cutting loop 110 has one or more sharpened and/or serrated edges. Oscillation and/or rotation may be incorporated into the tissue cutting device 100 in addition to the incorporation of any other form of energy. Oscillation and/or rotation is activated and deactivated by an oscillation/rotation controller (not shown) preferably located in the handle 190. The oscillation/rotation controller may be manually or automatically controlled. In one embodiment (not shown), the oscillation/rotation controller is automatically activated when the cutting loop is energized with a secondary form of energy (i.e. radio frequency energy).

The cutting loop 110 may one or multiple loops. The multiple loops of the cutting loop 110 may have similar or dissimilar properties, configurations and/or functions. In one embodiment (not shown), the cutting loop 110 is comprised of an outer and an inner loop. The inner loop is nested within the outer loop. Preferably the leading edges 118 and/or the trailing edges 117 of the inner and outer loops are serrated. The inner loop oscillates and/or rotates to cut tissue. The outer loop oscillates and/or rotates in an opposing direction to the inner loop which facilitates cutting by preventing the tissue from moving with the oscillation or rotation of the inner loop. In an alternative, the outer loop does not oscillate or rotate but the serrated leading edge 188 or trailing edge 177 still facilitates stabilization of the tissue depending on the direction of the cut.

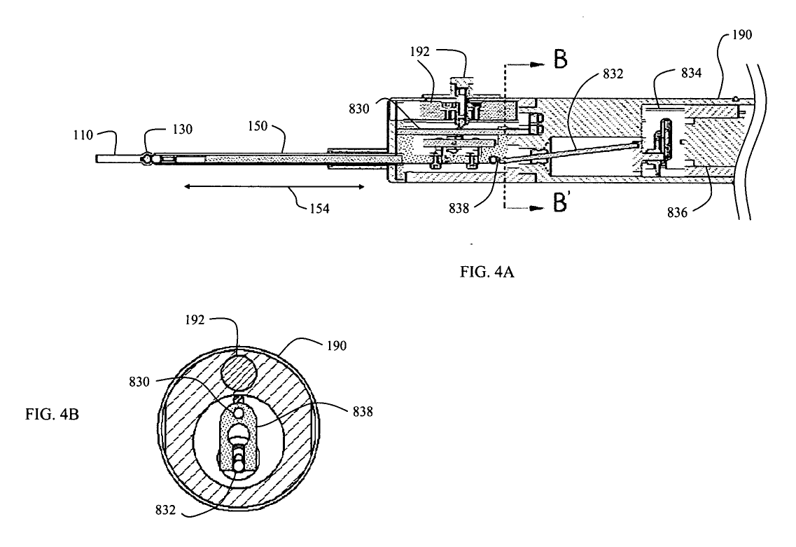

An exemplary embodiment illustrating a mechanism of oscillating the cutting loop 110 is shown in a cross-sectional side view in FIG. 4A, taken through the plane A-A′ in FIG. 2E, and a cross-sectional front view in FIG. 4B, taken through a plane B-B′ in FIG. 4A. A motor 836 located in the handle 190 is operatively coupled with a gear box 834. The configuration of the gear box 834 determines the peak-to-peak distance of oscillation of the cutting loop 110. The gear box 834 rotates a drive bar 832 that is operatively coupled to a rocking base 838 which is rotatable around a shaft 830 and is operatively coupled with the loop holder 130. Rotation of the drive bar 832 by the motor 836 oscillates the rocking base 838 which oscillates around the shaft 830. Oscillation of the rocking base 838 oscillates the loop holder 130 and cutting loop 110 in a plane that is generally orthogonal to the probe axis 154.

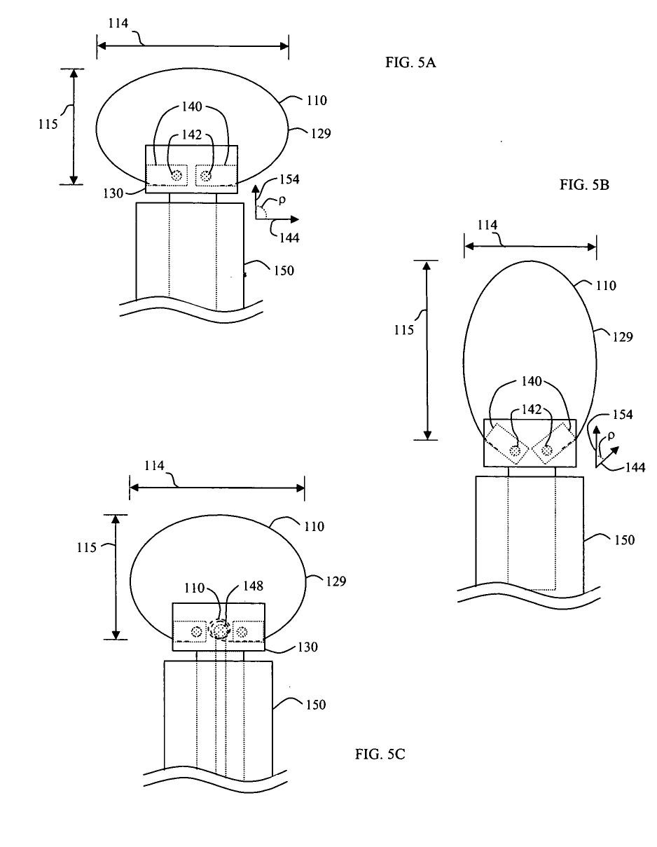

In a further embodiment illustrated in top views in FIGS. 5A-5C, the primary loop shape width 114 of the cutting loop 110 is variable or adjustable. The cutting loop 110 can be affixed to one or more width adjustors 140 that may be housed at least partially within the loop holder 130. The width adjustors 140 may pivot simultaneously or independently about pivot centers 142 which are preferably positioned within the width adjustors 140. The position of the pivot centers 142 within the width adjustors 140 preferably optimizes the pivot of the width adjustors 140. Pivoting of at least one of the width adjustors 140 may be controlled by a width controller (not shown) located on the handle 190. In an alternative (not shown), a primary width adjustor is pivotable and a secondary width adjustor is fixed and not pivotable. In a further alternative (not shown), one end of the cutting loop 110 is affixed to a width adjustor 140 and the other end of the cutting loop 110 is affixed to the loop holder 130. As shown in FIGS. 5A and 5B, a length of the width adjustors 140 defines a width adjustor axis 144. The relation of the width adjustor axis 144 to the probe axis 154 defines a width angle ρ. In FIG. 5A, the width adjustors 140 are rotated such that the width angle ρ is generally 90° which provides a larger primary loop shape width 114 and a smaller primary loop shape height 115, than in FIG. 5B, where width adjustors 140 are rotated such that the width angle ρ is less than 90°.

An exposed loop length 129, i.e., the length of the cutting loop 110 not housed within the loop holder 130, may be fixed as shown in FIGS. 5A and 5B. Alternatively, as shown in FIG. 5C, the exposed loop length 129 can be variable or adjustable. In particular, a length at one end of the cutting loop 110 may be wrapped around a rotatable coiler or winder 148 located in the loop holder 130 and/or the probe 150. As the coiler 148 is rotated, the exposed loop length 129, i.e., the length of the cutting loop 110 that is not coiled around the coiler 148, increases or decreases depending on the direction of rotation of the coiler 148. Increasing or decreasing the exposed loop length 129 increases or decreases the primary loop shape width 114 and/or the height 115. Although one rotatable coiler 148 is shown, two rotatable coilers may be provided to coil both ends of the cutting loop 110 and the rotatable coilers may operate cooperatively with or independently of each other. If the rotatable coilers operate cooperatively with each other, the rotatable coilers may rotate in opposite directions, i.e., clockwise and counterclockwise, so that both rotatable coilers are working toward decreasing or increasing the exposed loop length 129. The rotatable coilers may alternatively or additionally be configured to rotate in the same direction at the same or different rates such as to rotate and/or oscillate the cutting loop 110 in a plane generally orthogonal to the direction of the cut. In addition, the probe 150 may alternatively contain one or more rotatable coilers 148 and no width adjustors 140. The primary loop shape of the cutting loop 110 may have a fixed width 114 and height 115, a fixed width 144 and variable height 115, a variable width 114 and fixed height 115, or a variable width 114 and height 115.

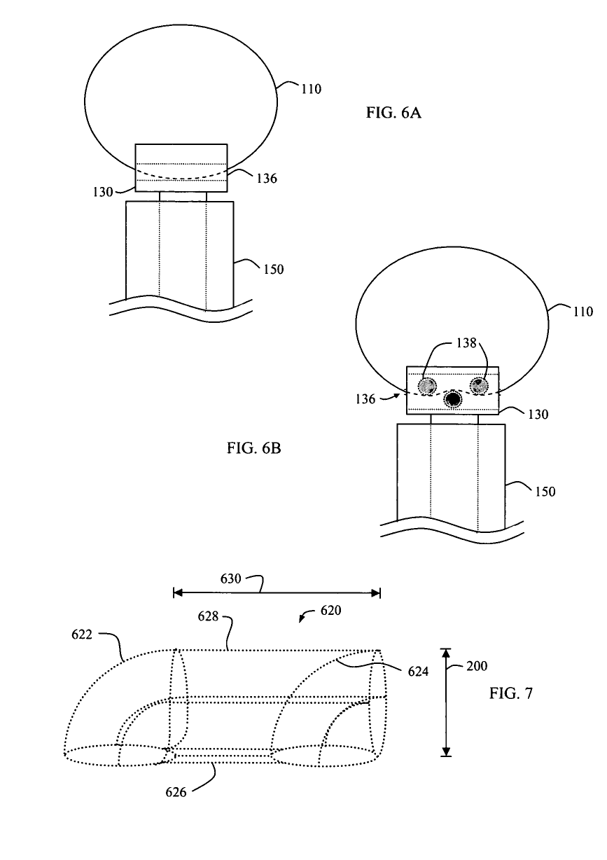

FIGS. 6A and 6B illustrate the cutting loop 110 and the loop holder 130 in more detail. As shown, the cutting loop 110 may be configured as a closed shape that passes through a loop holder channel 136 defined in the loop holder 130. The cutting loop 110 may be configured as any closed geometric or irregular shape. The loop holder 130 is rotatable so as to vary the loop angle θ (not shown). In the embodiment illustrated in FIG. 6B, one or more gears 138 housed within the loop holder 130 and/or the probe 150 can rotate and/or oscillate the cutting loop 110 in a plane preferably generally orthogonal to the direction of the cut. The orientation of the one or more gears 138 with respect to each other may be fixed or variable. The specific orientations of the one or more gears 138 may be determined depending on the desired primary loop shape 126, for example.

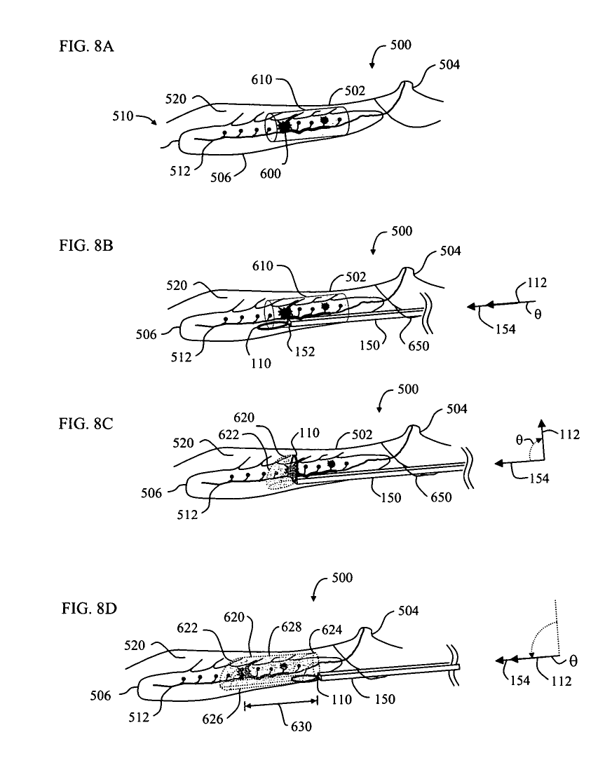

FIGS. 8A-8D are perspective sectional views of part of a breast 500. Deep to a skin surface 502 of the breast 500 is a lobe 506 that extends from a nipple/areolar complex 504 towards a periphery 510 of the breast 500. One or more main ducts, herein depicted as a main duct 512, extend generally along a length of the lobe 506. A lesion 600 is shown at least within part of the lobe 506. The lesion 600 may be an invasive cancer, an extension of the cancer in the main duct 512, in duct branches (not shown) and/or in Cooper's ligament(s) and/or any multifocal cancer. An estimated volume of tissue 610 to be excised that contains the lesion 600 as well as a margin of normal tissue surrounding the lesion 600 is shown in FIG. 8A. Although the estimated volume of tissue 610 contains part of the lobe 506 and part of a surrounding tissue 520, the estimated volume of tissue 610 may encompass almost all of a lobe 506, an entire lobe 506 or more than one lobe 506 of the breast 500 depending on the size and extent of the lesion 600 and the purpose of the procedure, e.g., biopsy or therapeutic excision. The lesion 600 is targeted using a medical targeting device (not shown). Preferably the medical targeting device is an imaging device such as a device for ultrasound imaging, magnetic resonance imaging, computerized tomography, positron emission tomography, and x-ray imaging. The imaging device may use analog and/or digital imaging technologies. The imaging device produces two-dimensional, three-dimensional and/or four-dimensional images. Preferably the imaging device images at least all of part of the lesion 600, the estimated volume of tissue 610 and the tissue cutting device 100. The medical targeting device is positioned adjacent to the skin 502, at a distance from the skin 502 and/or within the breast 500. When located in the breast 500, the medical targeting device may be attached to or incorporated in the tissue cutting device 100 or may be separate from the tissue cutting device 100. Preferably the medical targeting device is used to guide the procedure using the tissue cutting device 100. Although not shown, one or more locators may also be positioned at or near the distal end of the probe. The locators provide a different or enhanced method of identifying at least part of the probe 150 within the tissue, for example, using any suitable type of light emission. A locator sensor preferably located external to the skin may be utilized to detect and identify the position of the locator.

After the estimated volume of tissue 610 is determined, the breast 500 is prepared and local anesthetic may be administered using standard surgical technique. A skin incision 650 is made preferably using a surgical scalpel and preferably at a border of the nipple/areolar complex 504. The probe 150 is inserted through the skin incision 650 and positioned preferably under the estimated volume of tissue 610. In one embodiment (not shown), an introducer may be inserted into the breast 500 prior to insertion of the probe 150 to facilitate accurate positioning of the probe 150. The introducer may include, for example, a needle guide, a dilator and a sheath. The needle guide may be positioned under the estimated volume of tissue 610. After adequate positioning is determined, the dilator and sheath slide over the needle guide. The dilator enlarges a track around the needle guide and then the dilator and needle guide are removed, leaving the sheath in place. The probe 150 or preferably the probe cover 158 may be positioned at the end of the sheath outside of the breast 500. The probe 150 may then slide within the sheath and into the breast 500 until the distal end 152, the cutting loop 110, and/or the loop holder 130 is distal to the end of the sheath that is in the breast 500.

As shown in FIG. 8B, the probe 150 is positioned under the estimated volume of tissue 610 and the cutting loop 110 and loop holder 130 have advanced out of the distal end 152. The loop angle θ is generally 0°. The cutting loop 110 may be energized and rotated until the loop angle θ is generally 90° as shown in FIG. 8C. Cutting of tissue during the initial rotation of the cutting loop 110 creates a specimen start 622 of a specimen 620 of tissue. Alternatively, the cutting loop 110 may be rotated such that the loop angle θ is less or greater than 90° to provide a cut height 200 that is less than the loop height 115. After the cutting loop 110 is rotated to the desired loop angle θ, the probe 150 is retracted to move the cutting loop 110 toward the skin incision 650. This completes a circumferential separation of the specimen 620 from the breast 500 along the specimen length 630 as shown in FIG. 8D. The probe 150 is retracted until the cutting loop 110 is proximal to the estimated volume of tissue 610 relative to the skin incision 650 such that when the cutting loop 110 is at the loop angle θ of 0°, the cutting loop 110 is proximal to the estimated volume of tissue 610. The cutting loop 110 being proximal to the estimated volume of tissue 610 is then rotated to the loop angle θ of 0° to separate a specimen end 624 and complete separation of the specimen 620 from the breast 500.

In a further embodiment, a tissue collector (not shown) may be attached to the probe 150, the loop holder 130 and/or the cutting loop 110. The tissue collector may collect the specimen 620 during or after the cutting of the specimen 620.

As illustrated in FIG. 7, the specimen start 622 is generally convex in shape and the specimen end 624 is generally concave in shape such that the specimen 620 is asymmetric in shape, e.g., asymmetric along the probe axis. Furthermore, the specimen 620 has a deep surface 626 and a superficial surface 628. At least part of the deep surface 626 is a generally flat surface that is created by the introducer (not shown) or the probe 150 during insertion into the breast 500. The superficial surface 628 is created by the cutting loop 110 and is generally curved. The asymmetry of the specimen 620 helps to orient the specimen 620 relative to the breast 500 after the specimen 620 is removed from the breast 500 without use of tissue dyes or creation of burn marks on the specimen 620 using energy (e.g. radio frequency energy). Although one example of an asymmetric shape of the specimen 620 is shown and described, various other shapes, asymmetric or symmetric, may be created using different configurations of the cutting loop 110.

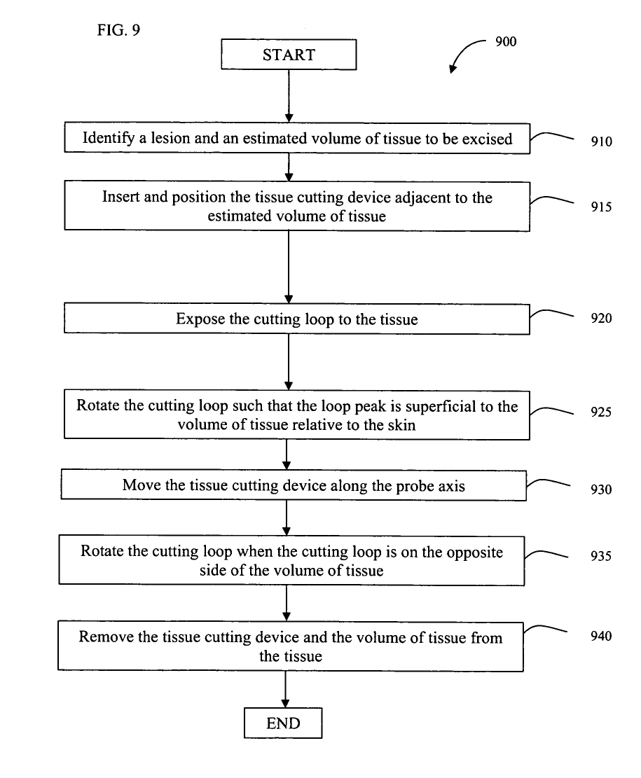

FIG. 9 is a flowchart illustrating a method 900 for removing a lesion in the breast using the tissue cutting device described above. The method begins at block 910 in which the lesion is identified and an estimated volume of tissue to be excised that contains at least part of the lesion for a biopsy or the entire lesion and a surrounding margin of normal tissue for a therapeutic procedure is determined. At block 915, the tissue cutting device with the cutting loop in the secondary loop shape is inserted through a skin incision into the breast tissue and positioned adjacent to the estimated volume of tissue such that when the entire leading edge of the cutting loop is exposed to the tissue, the loop peak is distal to the estimated volume of tissue relative to the skin incision.

The cutting loop is exposed to the tissue at block 920 and is energized and rotated preferably until the loop peak is superficial to the estimated volume of tissue relative to the skin surface at block 925. At block 930, the tissue cutting device is retracted to complete a circumferential cut along the length of the estimated volume of tissue. When the cutting loop is proximal to the volume of tissue relative to the skin incision, the cutting loop is rotated to 0° or 180° to complete the cutting of the volume of tissue at block 935. At block 940, the tissue cutting device and the volume of tissue are removed from the breast. In an alternative method (not shown), the cutting loop may be positioned proximal to the estimated volume of tissue and then rotated to a loop angle greater than 0° and less than 180°. The probe is then advanced to advance the cutting loop within the tissue. When the cutting loop is distal to the estimated volume of tissue, the cutting loop is rotated to the 0° or 180° position to complete the cutting of the specimen.

While the exemplary embodiments of the present invention are described and illustrated herein, it will be appreciated that they are merely illustrative and that modifications can be made to these embodiments without departing from the spirit and scope of the invention. Thus, the scope of the invention is intended to be defined only in terms of the following claims as may be amended, with each claim being expressly incorporated into this Description of Specific Embodiments as an embodiment of the invention.

1. A tissue cutting device, comprising:
a probe defining a probe axis;
a cutting loop configured to be in one of a storage configuration and a cutting configuration; and
a loop holder defining a loop holder axis generally orthogonal to the probe axis, the loop holder being configured to hold and to rotate the cutting loop about the loop holder axis when the cutting loop is in the cutting configuration so as to adjust a loop angle defined between the probe axis and the cutting loop.

2. The tissue cutting device of claim 1, wherein the probe includes a probe cover slidable along the probe axis and having a distal position in which the probe cover houses at least part of the loop holder and the cutting loop in the storage configuration and a proximal position in which at least part of the loop holder and the cutting loop are external to the probe cover and in which the cutting loop is in the cutting configuration.

3. The tissue cutting device of claim 1, wherein the cutting loop is configured in the storage configuration when retracted into the probe and when extended from a distal region of the probe, the cutting loop generally returning to a cutting configuration from the storage configuration.

4. The tissue cutting device of claim 1, further comprising a handle coupled to a proximal region of the probe, the handle housing a loop controller for at least one of selectively extending the cutting loop to the cutting configuration out of the probe and retracting the cutting loop to the storage configuration within the probe, and selectively rotating the loop holder and the cutting loop when the cutting loop is in the cutting configuration.

5. The tissue cutting device of claim 1, wherein the cutting loop has at least one of high elasticity, shape memory property and superelastic property.

6. The tissue cutting device of claim 1, wherein the cutting loop has a first edge and a second edge and wherein the first edge is one of longer than, equal in length to, and shorter than the second edge.

7. The tissue cutting device of claim 1, wherein the cutting loop has a first edge and a second edge and wherein at least one of the edges is at least one of pointed, flat, rounded, dull, sharpened, continuously serrated, intermittently serrated, regularly serrated, and irregularly serrated.

8. The tissue cutting device of claim 1, further comprising a loop width adjuster disposed in at least one of the loop holder and the probe, the loop width adjuster being configured to adjust a width of the cutting loop.

9. The tissue cutting device of claim 8, wherein the loop width adjuster is pivotable about a pivot, whereupon pivoting the loop width adjuster about the pivot adjusts the width of the cutting loop.

10. The tissue cutting device of claim 1, further comprising a loop length adjuster disposed in at least one of the loop holder and the probe, the loop length adjuster being configured to adjust a length of the cutting loop.

11. The tissue cutting device of claim 10, wherein the loop length adjuster includes a cutting loop winder configured to at least one of wind and unwind a length of the cutting loop to shorten and lengthen, respectively, the length of the cutting loop exterior to the loop holder.

12. The tissue cutting device of claim 1, wherein the cutting loop is fixedly attached to the loop holder.

13. The tissue cutting device of claim 1, further comprising a tissue collector coupled to at least one of the probe, the loop holder and the cutting loop.

14. The tissue cutting device of claim 13, wherein the tissue collector is adapted to collect tissue at least one of as the tissue is severed by the cutting loop and after the tissue is severed by the cutting loop.

15. The tissue cutting device of claim 1, further comprising an energy source operatively coupled to the cutting loop.

16. The tissue cutting device of claim 15, wherein energy provided by the energy source is selected from the group consisting of radio frequency, laser, ultrasound, heat, cold, oscillation, vibration, rotation, liquid pressure and gas pressure.

17. The tissue cutting device of claim 16, wherein the radio frequency energy source is configured to apply a current to the cutting loop and wherein the cutting loop is at least partially insulated to concentrate the current on a portion thereof.

18. The tissue cutting device of claim 16, wherein the rotation or oscillation is generally in a direction orthogonal to the probe axis.

19. The tissue cutting device of claim 18, further comprising at least one gear disposed in at least one of the loop holder and the probe, the at least one gear being configured to at least one of rotate and oscillate the cutting loop.

20. The tissue cutting device of claim 1, wherein the cutting loop includes a metallic material selected from the group consisting of a metal, a metal alloy, a metal laminate, and a metal composite.

21. The tissue cutting device of claim 20, wherein the metallic material is one of titanium, titanium alloy, nickel-titanium alloy, nickel-chromium alloy, chromium-nickel alloy, cobalt chromium-nickel alloy and iron-chromium alloy.

22. The tissue cutting device of claim 20, wherein the cutting loop includes at least one additional material to provide at least one of electrical insulation, heat insulation, electrical conductivity, heat conductivity, strength, lubricity, and sensor.

23. The tissue cutting device of claim 22, wherein the at least one additional material is selected from the group consisting of ceramics, polymers, plastics, metals, metal alloys, glass, diamonds, diamond-like carbon, and metal-doped diamond noncomposite coating, and nonmetal-doped diamond noncomposite coating.

24. The tissue cutting device of claim 1, wherein the probe includes at least one accessory channel.

25. The tissue cutting device of claim 24, wherein the at least one accessory lumen includes at least one of a transport lumen configured to transport a material to be to a distal end of the probe and a vacuum lumen operatively connected to a vacuum source.

26. The tissue cutting device of claim 1, wherein the cutting loop includes a plurality of loops.

27. The tissue cutting device of claim 26, wherein the plurality of loops of the cutting loop move relative to each other by at least one of rotating and oscillating.

28. A device, comprising a tissue cutting device configured to cut an asymmetric volume of tissue.

29. The device of claim 28, wherein the tissue cutting device includes a probe defining a probe axis, a cutting loop configured to be in one of a storage configuration and a cutting configuration, and a loop holder configured to hold and to rotate the cutting loop about a loop holder orthogonal to the probe axis when the cutting loop is in the cutting configuration, and wherein the asymmetric volume of tissue is cut by the loop holder rotating the cutting loop to adjust the loop angle upon returning the cutting loop from the storage configuration to the cutting configuration, by moving the tissue cutting device generally along the probe axis, and by the loop holder rotating the cutting loop again so the loop angle is approximately 0° to complete the cut of the asymmetric volume of tissue.

30. A tissue cutting method, comprising:
positioning a distal region of a probe of a tissue cutting device adjacent to a volume of tissue to be excised, the probe defining a probe axis;
returning a cutting loop to a cutting configuration from a storage configuration;
rotating a loop holder to rotate the cutting loop attached thereto about a loop holder axis defined by the loop holder, the loop holder axis being generally orthogonal to the probe axis, the rotating adjusts a loop angle defined between the probe axis and the cutting loop; and
moving the tissue cutting device such that the cutting loop cuts the volume of tissue.

31. The tissue cutting method of claim 30, wherein returning the cutting loop to the cutting configuration from the storage configuration includes extending the cutting loop from a distal region of the probe.

32. The tissue cutting method of claim 30, further comprising:
identifying a lesion; and
estimating the volume to tissue to be excised based on the identified lesion;

33. The tissue cutting method of claim 30, wherein the rotating positions the cutting loop to at least partially encircle the volume of tissue.

34. The tissue cutting method of claim 30, wherein the moving is along the probe axis.

35. The tissue cutting method of claim 30, further comprising after the moving, rotating the loop holder about the loop holder axis to rotate the cutting loop to complete cutting of the volume of tissue.

36. The tissue cutting method of claim 30, wherein at least one of the extending and rotating is via a loop controller on a handle coupled to a proximal region of the probe.

37. The tissue cutting method of claim 30, wherein the cutting loop has at least one of shape memory property, superelastic property, and high elasticity.

38. The tissue cutting method of claim 30, wherein the cutting loop has a first edge and a second edge and wherein the first edge is one of longer than, equal in length to, and shorter than the second edge.

39. The tissue cutting method of claim 30, wherein the cutting loop has a first edge and a second edge and wherein at least one of the edges is at least one of pointed, flat, rounded, dull, sharpened, continuously serrated, intermittently serrated, regularly serrated, and irregularly serrated.

40. The tissue cutting method of claim 30, further comprising adjusting a width of the cutting loop after the extending.

41. The tissue cutting method of claim 40, wherein the cutting loop width adjusting includes pivoting a loop width adjuster about a pivot.

42. The tissue cutting method of claim 30, further comprising adjusting a length of the cutting loop length exterior to the loop holder after the extending.

43. The tissue cutting method of claim 42, wherein the cutting loop length adjusting includes at least one of winding and unwinding the cutting loop onto and off of a cutting loop winder.

44. The tissue cutting method of claim 30, wherein the cutting loop is fixedly attached to the loop holder.

45. The tissue cutting method of claim 30, further comprising collecting the volume of tissue in a tissue collector coupled to at least one of the probe, the loop holder and the cutting loop.

46. The tissue cutting method of claim 45, wherein the collecting is at least one of during the moving of the tissue cutting device and after the moving of the tissue cutting device.

47. The tissue cutting method of claim 30, further comprising applying an energy to the cutting loop.

48. The tissue cutting method of claim 47, wherein the energy is selected from the group consisting of radio frequency, laser, ultrasound, heat, cold, oscillation, vibration, rotation, liquid pressure and gas pressure.

49. The tissue cutting method of claim 48, further comprising applying a radiofrequency current to the cutting loop, wherein the cutting loop is at least partially insulated to concentrate the radiofrequency current on a portion thereof.

50. The tissue cutting method of claim 48, further comprising at least one of rotating and oscillating the cutting loop by actuating a gear coupled to the cutting loop.

51. The tissue cutting method of claim 30, wherein the cutting loop includes an electrically conductive material.

52. The tissue cutting method of claim 51, wherein the electrically conductive material is a metallic material selected from the group consisting of a metal, a metal alloy, a metal laminate, and a metal composite.

53. The tissue cutting method of claim 52, wherein the metallic material is one of titanium, titanium alloy, nickel-titanium alloy, nickel-chromium alloy, and iron-chromium alloy.

54. The tissue cutting method of claim 30, further comprising delivering a material to a distal region of the probe via an accessory lumen of the probe.

55. The tissue cutting method of claim 30, further comprising applying vacuum to a distal region of the probe via a vacuum lumen of the probe operatively coupled to a vacuum source.

56. The tissue cutting method of claim 30, wherein the volume of tissue is an asymmetric volume of tissue.

57. The tissue cutting method of claim 30, further comprising:
rotating the loop holder after the moving to rotate the cutting loop about the loop holder axis so that the loop angle is approximately 0° or 180° to complete the cut of the asymmetric volume of tissue.

58. The tissue cutting method of claim 30, further comprising, during the moving the tissue cutting device, moving a plurality of loops of the cutting loop relative to each other, the moving the plurality of loops being at least one of rotating and oscillating.

59. The tissue cutting method of claim 30, wherein the returning the cutting loop to the cutting configuration from the storage configuration includes sliding a probe cover of the probe in a proximal direction from a distal position in which the probe cover houses at least part of the loop holder and the cutting loop in the storage configuration to a proximal position in which the cutting loop extends from a distal end of the probe cover returns to the cutting configuration.

60. The tissue cutting method of claim 59, further comprising:
positioning a sheath in the tissue;
engaging a proximal end of the sheath to a distal end of the probe cover; and
pushing at least part of the probe through a distal region of the probe cover and into the sheath until at least the cutting loop is distal to a distal end of the sheath and the cutting loop returns to the cutting configuration.

61. The tissue cutting method of claim 60, further comprising:
positioning a distal end of a guide adjacent to the volume of tissue;
enlarging a track in the tissue around the guide by sliding a dilator and the sheath over the guide; and
removing at least the dilator, leaving at least the sheath in place.
